(19) United States
(12) Patent Application Publication
(10) Pub. No.: US 2021/0330821 A1
(43) Pub. Date: Oct. 28, 2021

(54) METHODS OF MAKING PROSTATE CANCER TREATMENT DECISIONS

(71) Applicant: Progenics Pharmaceuticals, Inc, New York, NY (US)

(72) Inventor: Vivien Wong, Scarsdale, NY (US)

(73) Assignee: Progenics Pharmaceuticals, Inc, New York, NY (US)

(21) Appl. No.: 17/227,048

(22) Filed: Apr. 9, 2021

Related U.S. Application Data
(63) Continuation of application No. PCT/US2019/055946, filed on Oct. 11, 2019.
(60) Provisional application No. 62/842,136, filed on May 2, 2019.
(60) Provisional application No. 62/804,081, filed on Feb. 11, 2019.
(60) Provisional application No. 62/754,520, filed on Nov. 1, 2018.
(60) Provisional application No. 62/744,400, filed on Oct. 11, 2018.

(51) Int. Cl. A61K 51/04; A61P 35/04

(52) U.S. Cl. CPC A61K 51/0455; A61P 35/04

(57) ABSTRACT

Provided herein are methods for assessing a subject with prostate cancer. The methods are also directed to or may further comprise treating the subject and/or making treatment management decisions based on the assessing.

### RELATED APPLICATIONS

This application is a continuation of International Application No. PCT/US2019/055946, filed Oct. 11, 2019, which claims the benefit of priority under 35 U.S.C. § 119 to U.S. Provisional Application No. 62/744,400 filed Oct. 11, 2018, U.S. Provisional Application No. 62/754,520 filed Nov. 1, 2018, U.S. Provisional Application No. 62/804,081 filed Feb. 11, 2019 and U.S. Provisional Application No. 62/842,136 filed May 2, 2019, the entire contents of each of which are incorporated herein by reference.

### BACKGROUND OF THE INVENTION

Prostate cancer is the second most common form of cancer affecting men in the United States: an estimated one in seven men will be diagnosed with prostate cancer in his lifetime. The American Cancer Society estimates that each year approximately 164,609 new cases of prostate cancer will be diagnosed, and about 26,730 men will die of the disease. Approximately 2.9 million men in the U.S. currently count themselves among prostate cancer survivors. New methods are needed to expand treatment and treatment management options.

### SUMMARY OF THE INVENTION

In one aspect, a method of treating, or making a treatment management decision for, prostate cancer in a subject, the method comprising determining the presence or absence of one or more metastases in the subject with ¹⁸F-DCFPyL PET/CT is provided. In one embodiment of such methods, the method further comprises treating or making a treatment management decision for the subject based on the presence or absence of one or more metastases. In one embodiment of any one of the methods provided herein, the determining is based on obtaining or being given the results of any one of the methods of assessment provided herein. In one embodiment of any one of the methods provided herein, the determining is based on performing any one of the methods of assessment provided herein.

In one embodiment of any one of the methods provided herein, the subject is any one of the subjects provided herein, such as one with metastatic or recurrent prostate cancer, or is suspected of having metastatic or recurrent prostate cancer. In one embodiment of any one of the methods provided herein, the subject is one with extra-prostatic lesions, or is suspected of having extra-prostatic lesions. In one embodiment of any one of the methods provided herein, the subject is one with pelvic (e.g., lymph node) metastases, or is suspected of having pelvic (e.g., lymph node) metastases. In one embodiment of any one of the methods provided herein, the subject is one with distant metastases, or is suspected of having distant metastases. In one embodiment of any one of the methods provided herein, the subject is one with nodal, bone, and/or visceral/soft tissue metastases.

In one embodiment of any one of the methods provided herein, the one or more of the metastases is greater than 4 mm. In one embodiment of any one of the methods provided herein, the median lymph node metastases >4 mm.

In one embodiment of any one of the methods provided herein, ¹⁸F-DCFPyL is administered 1-2 hours prior to PET/CT to the subject.

In one embodiment of any one of the methods provided herein, 9 mCi (333 MBq) of ¹⁸F-DCFPyL is administered by IV injection to the subject.

In one embodiment of any one of the methods provided herein, the method further comprises treating the subject based on the determining. In one embodiment of any one of the methods provided herein, the treatment is any one of the treatments provided herein.

In one embodiment of any one of the methods provided herein, the method further comprises making a treatment management decision. In one embodiment of any one of the methods provided herein, the making a treatment management decision includes any one or more of the treatment management decisions provided herein.

In one embodiment of any one of the methods provided herein, the subject has undergone a prior diagnostic test, such as a PSA test or a conventional imaging test. In one embodiment of any one of the methods provided herein, the prior diagnostic test was negative or equivocal findings for prostate cancer on conventional imaging.

In one embodiment of any one of the methods provided herein, the method further comprises a step of performing an additional diagnostic test on the subject, such as a PSA test or conventional imaging test. In one embodiment of any one of the methods provided herein, the PSA test result was detectable or rising PSA post-radical prostatectomy in the subject. In one embodiment of any one of the methods provided herein, the PSA test result was an increase in PSA level post-radiation therapy, cryotherapy, or brachytherapy in the subject.

### BRIEF DESCRIPTION OF THE DRAWINGS

FIG. 1 shows the OSPREY study design and cohorts.

FIG. 2 shows the diagnostic performance in pelvic lymph nodes.

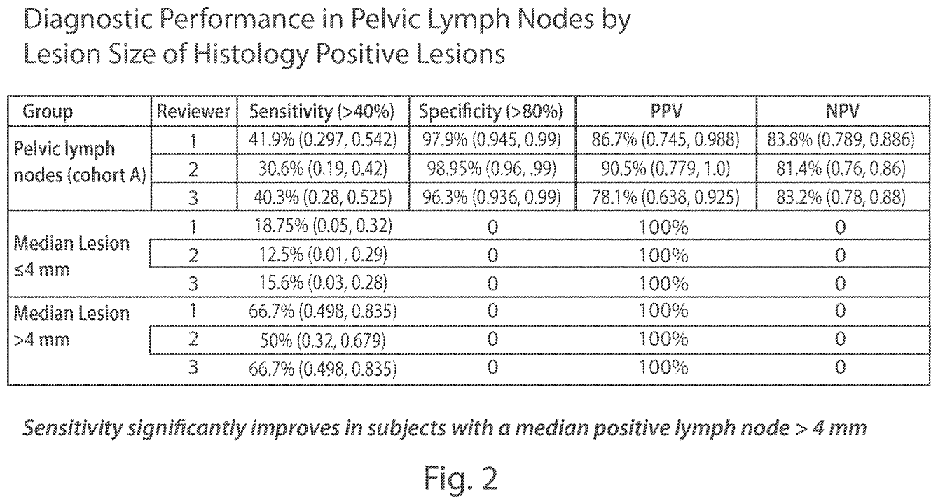

### DETAILED DESCRIPTION OF THE INVENTION

¹⁸F-DCFPyL, also referred to herein PyL, is a fluorinated PSMA-targeted Positron Emission Topography/Computed Tomography (“PET/CT”) imaging agent that enables visualization of metastases, such as bone and soft tissue metastases. It has been surprisingly found that imaging with such an agent can be used to determine the presence or absence of recurrent or metastatic prostate cancer with a high level of sensitivity or positive predictive value of subjects as provided herein.

A subject provided herein is one with, or is suspected of having, metastatic or recurrent prostate cancer on which PSMA is expressed. PSMA is a 100 kD Type II membrane glycoprotein expressed in prostate tissues (Horoszewicz et al., 1987, Anticancer Res. 7:927-935; U.S. Pat. No. 5,162,504). PSMA was characterized as a type II transmembrane protein having sequence homology with the transferrin receptor (Israeli et al., 1994, Cancer Res. 54:1807-1811) and with NAALADase activity (Carter et al., 1996, Proc. Natl. Acad. Sci. U.S.A. 93:749-753). PSMA is expressed in increased amounts in prostate cancer (Horoszewicz et al., 1987, Anticancer Res. 7:927-935; Rochon et al., 1994, Prostate 25:219-223; Murphy et al., 1995, Prostate 26:164-168; and Murphy et al., 1995, Anticancer Res. 15:1473-1479).

Also surprisingly found, in some embodiments, the identification of pelvic lymph node metastases of greater than 4 mm provided even more predictive power and sensitivity. Thus, in any one of the methods provided herein, the subject is one with (or is suspected of having) metastases, such as pelvic lymph node metastases, that are greater than 4 mm.

“Metastatic”, as used herein, refers to cancer, that has spread from the primary cancer or primary cancer site. In prostate cancer, the metastases can be extra-prostatic. The metastases can occur within the pelvis, such as in the pelvic lymph nodes, or can be in a site distant from the pelvis (also referred to herein “distant metastases”). In one embodiment, of any one of the methods provided herein, the metastases are pelvic lymph node metastases or metastases in other distant sites.

“Recurrent”, as used herein, refers to prostate cancer that occurs after an initial prostate cancer has occurred or refers to prostate cancer cells that have survived a treatment.

In one aspect, a method of treating or making a treatment management decision for any one of the subjects provided herein comprising identifying metastases in the subject with ¹⁸F-DCFPyL PET/CT imaging and treating or making a treatment management decision for the subject based on the presence or absence of identified metastases is provided.

“Treating, as used herein, refers to any clinical action taken or recommended to be taken to reduce or eliminate prostate cancer in a subject or to provide any benefit to the subject such as the reduction of symptoms as a result of having prostate cancer. Treatment of prostate cancer includes, but is not limited to, surgery, radiation, cryotherapy, brachytherapy, chemotherapy, or hormonal/androgen deprivation therapy (ADT) therapy. In any one of the methods provided herein, the treating or treatment of the subject can comprise any one of the forms of treatment provided herein or a recommendation to provide such a treatment to a subject.

In one embodiment of any one of the methods provided herein, treating or treatment of a subject comprises I-131 1095 in combination with enzalutamide. Small molecule therapeutic, I-131 (iodine-131) 1095, binds to the extracellular domain of PSMA. Preclinical data has shown high tumor uptake and a favorable tumor to kidney discrimination yielding a lethal radiation dose to the tumor while minimizing normal tissue dose. In human prostate cancer mouse models, the compound, administered in single or multiple dose schedules, significantly reduced tumor burden for a prolonged period of time and enhanced survival with no significant signs of toxicity. When used in a compassionate use setting, I-131 1095 markedly reduced PSA levels and bone pain but was well tolerated in a group of heavily-pretreated advanced prostate cancer patients.

The chemical structure of I-131 1095 (or 131I 1095) (i.e., 131I-(S)-2-(3-((S)-1-carboxy-5-(3-(4-iodophenyl)ureido)pentyl)ureido)pentanedioic acid) is:

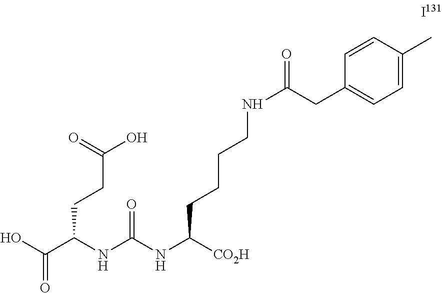 [SMILES: Cc1ccc(CC(=O)NCCCC[C@H](NC(=O)N[C@@H](CCC(=O)O)C(=O)O)C(=O)O)cc1]

Enantiomers, stereoisomers, rotamers, tautomers, diastereomers, or racemates thereof are also included in the definition of “I-131 1095”. U.S. Pat. No. 8,487,129 describes such compounds, which compounds and methods of their making are incorporated herein by reference. These compounds are for use in any one of the methods and compositions provided herein, in an embodiment.

“Making a treatment management decision”, as used herein, refers to any decision a clinician may make for a subject with prostate cancer in order to monitor and/or treat the prostate cancer in the subject. Treatment management decisions include, but are not limited to, determining that a biopsy be performed, changing the location to perform a biopsy, changing the frequency of performing a biopsy, determining that a surgery be performed, changing the type of surgery to be performed, changing the location to perform a surgery, changing the timing of performing a surgery, determining that radiation be administered, changing the type of radiation to administer, determining the dose of radiation to administer, determining the location to which to administer radiation, changing the dose of radiation to administer, changing the location to which to administer radiation, determining that chemotherapy be performed, changing the type of chemotherapy to administer, determining the dose of chemotherapy to administer, changing the dose of chemotherapy to administer, changing the regimen for administering chemotherapy, determining hormonal/ADT therapy be administered, changing the type of hormonal/ADT therapy to administer, determining the dose of hormonal/ADT therapy to administer, changing the dose of hormonal/ADT therapy to administer, and changing the regimen for administering hormonal/ADT therapy. In any one of the methods provided herein, the step of making a treatment management decision can include any one or more of the foregoing. In any one of the methods provided herein, the method may further comprise a step of treating or monitoring the subject (or recommending the treatment or monitoring to the subject) according to the treatment management decision.

The subject of any one of the methods provided herein can be one with metastatic or recurrent prostate cancer (or can be one in which metastatic or recurrent prostate cancer is suspected).

In some embodiments of any one of the methods provided, the subject may have a high or rising prostate-specific antigen (PSA) levels, such as one with elevated PSA that is greater than or equal to 0.2 ng/mL above the nadir. In some embodiments of such subjects, the subject is one that has undergone radiation therapy, cryotherapy or brachytherapy. In some embodiments of any one of the methods provided herein, the subject is one who has undergone radiation therapy, cryotherapy or brachytherapy or the method includes a step of treating the subject with radiation therapy, cryotherapy or brachytherapy.

In some embodiments of any one of the methods provided, the subject may have a high or rising prostate-specific antigen (PSA) levels, such as one with detectable or rising PSA that is greater than or equal to 0.2 ng/mL with a confirmatory PSA of greater than or equal to 0.2 ng/mL. In some embodiments of such subjects, the subject may have undergone a radical prostatectomy. In some embodiments of any one of the methods provided herein, the subject is one who has undergone radical prostatectomy or the method includes a step of treating the subject with radical prostatectomy.

In some embodiments of any one of the methods provided, the subject may have negative or equivocal findings for prostate cancer with conventional imaging, such as conventional imaging performed as part of a standard of care workup. Conventional imaging methods include, but are not limited to pelvic CT/MRI, whole-body scan, NaF, fluciclovine or choline PET. In some embodiments of any one of the methods provided herein, the subject is one who has undergone conventional imaging or the method includes a step of assessing the subject with conventional imaging.

In some embodiments of any one of the methods provided herein, the subject is one who has undergone PSA level testing or the method includes a step of testing the PSA level in the subject.

In one embodiment of any one of the methods provided herein, a subject has had prior antiandrogen therapy, such as with abiraterone. In another embodiment of any one of the methods provided herein, such subject has had prior antiandrogen therapy, such as with abiraterone, but not prior cytotoxic chemotherapy, such as with taxane chemotherapy. In another embodiment of any one of the methods provided herein, any one of such subjects has prostate cancer that has progressed despite these prior treatment(s). In another embodiment of any one of the methods provided herein, any one of such subjects is one with mCRPC that has progressed despite prior treatment(s).

In one embodiment of any one of the methods provided herein, a subject has had multiple rounds of prior antiandrogen therapy, such as with abiraterone. In another embodiment of any one of the methods provided herein, a subject has had prior antiandrogen therapy, such as with abiraterone, but not prior cytotoxic chemotherapy, such as with taxane chemotherapy. In another embodiment of any one of the methods provided herein, any one of such subjects has prostate cancer that has progressed despite the prior treatment(s). In another embodiment of any one of the methods provided herein, any one of such subjects is one with mCRPC that has progressed despite prior treatment(s).

An “antiandrogen,” as used herein, refers to an agent that blocks (e.g., inhibits) the action of androgen hormones and androgen-regulated molecules. Adrenergic receptor antagonists are herein considered to be antiandrogens. The term “antiandrogen” includes antiandrogens, antiandrogen analogs, and antiandrogen derivatives. In prostate cancer, antiandrogens block the activity of testosterone, which typically slows prostate cancer growth. In some embodiments, an antiandrogen blocks enzyme cytochrome P450 17A1, encoded by the CYP17A gene. Antiandrogens may be steroidal or non-steroidal (also referred to as “pure”). Examples of antiandrogens include, without limitation, abiraterone (ZYTIGA®), enzalutamide (XTANDI®), nilutamide (NILANDRON®), flutamide (EULEXIN®), bicalutamide (CASODEX®), and orteronel (TAK-700, Tokai Pharmaceuticals, Inc.)

“Progression”, as used herein, refers to prostate cancer cell proliferation that is not reduced, such as with treatment, such as with any one of the prior treatments or combinations thereof that are referred to herein, respectively. Disease progression may be indicated by rising PSA levels (e.g., an increase from baseline or a prior measurement of ≥25% and ≥2 ng/mL above nadir with or without a second such assessment of progression ≥3 weeks later), soft tissue disease progression as defined by RECIST 1.1, bone disease progression defined by two or more new lesions on bone scan, and/or new pain in an area of radiographically evident disease. In one embodiment of any one of the methods provided herein, prostate cancer that is progressing is not substantially inhibited by the prior treatment or combination thereof and would be considered non-responsive by a clinician.

In one embodiment of any one of the methods provided herein, the subject has or has had soft tissue or bone progression, such as with a scan that shows progression relative to a comparison scan performed during prior abiraterone therapy or after discontinuation from abiraterone. In one embodiment of any one of the methods provided herein, the subject has or has had soft tissue or bone progression, such as with a scan that shows progression relative to results from a previous scan, such as performed during prior abiraterone therapy or after discontinuation from abiraterone.

### EXAMPLES

### Example 1

### A PrOspective Phase 2/3 Multi-Center Study of ¹⁸F-DCFPyL PET/CT Imaging in Patients with PRostate Cancer—Examination of Diagnostic AccuracY (OSPREY)

### Background

Prostate-specific membrane antigen (PSMA) is a transmembrane protein that is overexpressed by prostate cancer (PCa) cells and can be targeted using the novel PET radiotracer ¹⁸F-DCFPyL. This example was aimed to determine the diagnostic performance of ¹⁸F-DCFPyL PET/CT for detecting pelvic lymph node metastases and other distant sites of disease in men with PCa.

### Methods

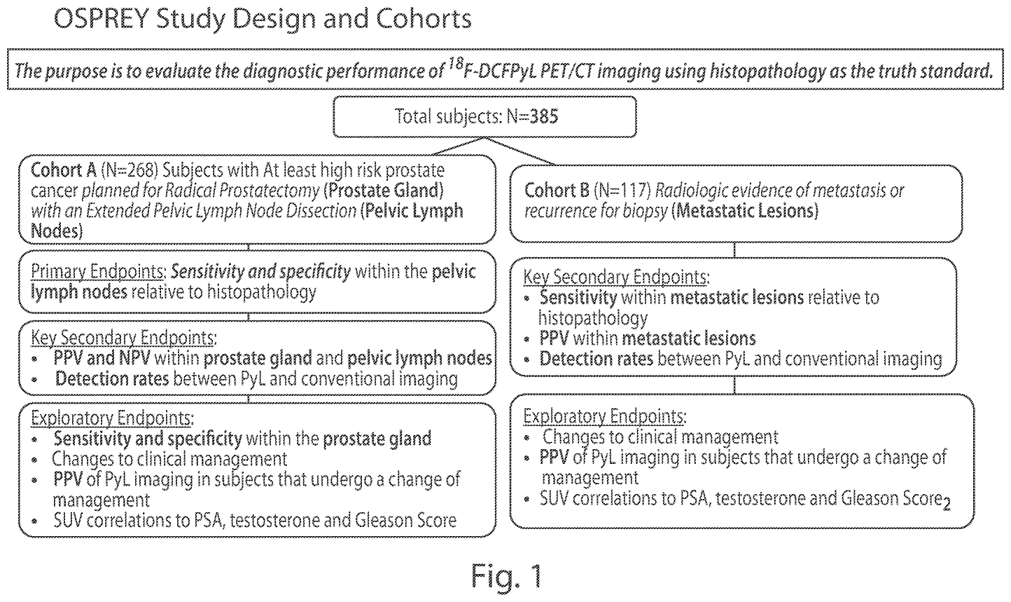

¹⁸F-DCFPyL PET/CT was evaluated in 385 men with high-risk PCa planned for radical prostatectomy and lymphadenectomy (Cohort A, n=268) or radiological evidence of metastatic PCa feasible for biopsy (Cohort B, n=117). 9 mCi (333 MBq) of ¹⁸F-DCFPyL was administered 1-2 hours prior to PET/CT. The coprimary endpoints of sensitivity and specificity of ¹⁸F-DCFPyL PET/CT for detecting pelvic lymph node metastases were evaluated in Cohort A. Secondary endpoints included safety in both cohorts, positive predictive value (PPV) and negative predictive value (NPV) in Cohort A, and sensitivity and PPV in Cohort B. Three central, blinded, and independent readers evaluated the ¹⁸F-DCFPyL scans. Histopathology served as the reference standard to which imaging findings were compared. FIG. 1 provides information regarding the study design and cohorts.

### Results

In Cohort A (n=252 evaluable), the sensitivity of ¹⁸F-DCFPyL PET/CT ranged among the three readers from 30.6-41.9% (lower bound of 95% CI: 19.2-29.7%), with a range of specificities of 96.3-98.9% (lower bound of 95% CI: 93.6-96.0%). Additionally, the PPV and NPV ranged from 78.1-90.5% and 81.4-83.8%, respectively. When the analysis was restricted to median lymph node metastases >4 mm, there was a marked improvement in sensitivity (range 51.7-65.5%; lower bound of 95% CI: 33.5-48.0%). In patients with distant metastatic PCa (Cohort B, n=93 evaluable), the values of sensitivity and PPV ranged from 92.9-98.6% (lower bound of 95% CI: 84.0-91.6%) and 81.2-87.8%, respectively. No drug-related serious adverse events were observed and 27 (7.0%) men experienced ≥1 drug-related adverse event, with dysgeusia (2.1%) and headache (2.1%) being most common.

### Diagnostic Performance Summary (Evaluable Set)

Pelvic Lymph Node Diagnostic Summary
The first co-primary endpoint of specificity ranged from 96-99% and was statistically significant at the pre-specified limit of 80%
The second co-primary endpoint of sensitivity ranged from 31-42% and did not meet the pre-specified confidence limit of 40%
The high PPV (78-91%) and NPV (81-84%) performance demonstrate strong clinical utility of ¹⁸F-DCFPyL PET/CT giving physicians the confidence to rely on the test result

Metastatic Diagnostic Summary
Secondary efficacy endpoint that represents recurrent or metastatic disease outside the pelvis (e.g., bone, soft tissue, and lymph nodes)
In this patient population, high sensitivity (93-99%) and PPV (81-88%) offers strong clinical utility to accurately detect distant metastatic lesions

Extra-Prostatic Diagnostic Summary
The performance of ¹⁸F-DCFPyL to detect extra-prostatic lesions, including the pelvic lymph nodes from extended pelvic lymph node dissection and distant metastatic or recurrent lesions from targeted biopsy, were statistically significant across all diagnostic parameters
This pooled analysis improves on the overall sensitivity of PyL to detect metastatic lesions (both pelvic and distant)

Cohort A and B

Prostate Gland Summary
This is an exploratory endpoint that demonstrates PyL is detecting prostate cancer with near perfect sensitivity and PPV
Confirming these are high risk prostate cancer patients
   Change of Medical Management of Subject Care (defined as, for example, change in one or more of the following):

TABLE 1
Parameter

Any Change in Management

Yes
No

Change in Diagnostic biopsy per standard of care

Yes
No

Change in surgery or type of Surgery Planned

Yes
No

Change in planned radiation

Yes

Change in Dose of radiation
Change in Location of radiation

No

Change in planned chemotherapy based on:

Yes

Change in Dose of chemotherapy
Change in Regimen of chemotherapy

No

Change in planned hormonal/ADT therapy

Yes

Change in Dose of hormonal/ADT therapy
Change in Regimen of hormonal/ADT therapy

No

### Change in Management Based on Sequential Review of Centrally Read Pre-PyL Imaging and Clinical Characteristics Followed by Re-Review Based on PyL Imaging Results

In one example, a change of management appeared to be indicated in ˜43% of subjects after reviewing the PyL PET/CT image with the most significant change to planned hormonal therapy

Cohort A

### Conclusions

¹⁸F-DCFPyL PET/CT was well tolerated and demonstrated high overall diagnostic performance in the detection of pelvic lymph node metastases and other distant sites of metastatic disease, as evidenced by its high specificity and PPV. These data suggest that ¹⁸F-DCFPyL PET/CT can enable more accurately informed treatment choices in men with prostate cancer.

### Example 2

### Study of ¹⁸F-DCFPyL PET/CT Imaging in Patients with Suspected Recurrence of Prostate Cancer (CONDOR)

TABLE 2
Arms and Interventions

| Arms | Assigned interventions |
|---|---|
| Experimental: 18F-DCFPyL | Drug: 18F-DCFPyE |
| Injection 9 mCi (333 MBq) IV | A single dose of 9 mCi (333 MBq) IV |
| injection of 18F-DCFPyE | injection of 18F-DCFPyE |
| | Other Names: |
| | PyL |

### Outcome Measures

Primary Outcome Measure:

1. Correct Localization Rate (CLR), defined as percentage of subjects with a one-to-one correspondence between localization of at least one lesion identified on ¹⁸F-DCFPyL PET/CT imaging and the composite truth standard, defined as either evaluable histopathology, informative correlative imaging, or PSA response after radiation therapy.

Within 60 days following PyL PET/CT imaging, either biopsy/surgery, conventional imaging, or locoregional radiation therapy of the PyL-suspected lesion(s) will be performed.

Secondary Outcome Measure:

2. Percentage of subjects with a change in intended prostate cancer treatment plans due to ¹⁸F-DCFPyL PET/CT imaging results.

The change in intended prostate cancer treatment plan will be based on Medical Management Questionnaires completed prior to and after PyL PET/CT imaging.

### Eligibility

Minimum Age: 18 Years

Maximum Age:

Sex: Male

Gender Based: Yes-only males subjects will be enrolled in this study.

Accepts Healthy Volunteers: No

Criteria: Inclusion Criteria:

Male >/=18 years of age

Histopathologically confirmed prostate adenocarcinoma per original diagnosis, with subsequent definitive therapy

Suspected recurrence of prostate cancer based on rising PSA after definitive therapy on the basis of:

a. Post-radical prostatectomy: Detectable or rising PSA that is ≥0.2 ng/mL with a confirmatory PSA ≥0.2 ng/mL (American Urological Association [AUA]); or

b. Post-radiation therapy, cryotherapy, or brachytherapy: Increase in PSA level that is elevated by ≥2 ng/mL above the nadir (American Society for Therapeutic Radiology and Oncology [ASTRO]-Phoenix)

Negative or equivocal findings for prostate cancer on conventional imaging performed as part of standard of care workup within 60 days prior to Day 1

Conventional imaging findings per institutional standard of care (SOC scan(s) (e.g., pelvic CT/MRI, whole-body bone scan, NaF, fluciclovine or choline PET) completed within 60 days prior to Day 1. SOC scan(s) performed more than 60 days prior to Day 1 may be repeated as a study Screening procedure and reviewed by the investigator prior to Day 1.

Life expectancy ≥6 months as determined by the investigator

Able and willing to provide informed consent and comply with protocol requirements

### Exclusion Criteria:

Subjects administered any high energy (>300 KeV) gamma-emitting radioisotope within five (5) physical half-lives prior to Day 1

Ongoing treatment with any systemic therapy (e.g., ADT, antiandrogen, GnRH, LHRH agonist or antagonist) for prostate cancer

Treatment with ADT in the past 3 months of Day 1

Receipt of investigational therapy for prostate cancer within 60 days of Day 1

Subjects with any medical condition or other circumstances that, in the opinion of the investigator, compromise the safety or compliance of the subject to produce reliable data or completing the study.

### Example 3

### Results from the OSPREY Trial: A PrOspective Phase 2/3 Multi-Center Study of ¹⁸F-DCFPyL PET/CT Imaging in Patients with PRostate Cancer—Examination of Diagnostic AccuracY

### Background

Prostate-specific membrane antigen (PSMA) is a transmembrane protein that is overexpressed by prostate cancer (PCa) cells. PSMA-based imaging is considered highly promising for PCa detection and can be targeted using the novel PET radiotracer ¹⁸F-DCFPyL. This prospective multicenter trial was designed to determine the safety and diagnostic performance of ¹⁸F-DCFPyL PET/CT for detecting pelvic lymph node metastases and sites of distant metastases in men with PCa.

### Methods

¹⁸F-DCFPyL PET/CT was evaluated in 385 men with high-risk PCa who were planned for radical prostatectomy with lymphadenectomy (Cohort A) or with radiological evidence of recurrent or metastatic PCa who were planned for biopsy (Cohort B). 9 mCi (333 MBq) of ¹⁸F-DCFPyL was administered 1-2 hours prior to PET/CT. Co-primary endpoints of specificity and sensitivity of ¹⁸F-DCFPyL PET/CT for detecting prostate cancer metastases in pelvic lymph nodes were evaluated in Cohort A. Key secondary endpoints included incidence of adverse events, positive predictive value (PPV) and negative predictive value (NPV) of ¹⁸F-DCFPyL PET/CT imaging to detect pelvic lymph node metastases in Cohort A, sensitivity and PPV of ¹⁸F-DCFPyL PET/CT imaging to detect prostate cancer within sites of metastasis or recurrence in Cohort B. Three central, blinded, and independent readers evaluated the ¹⁸F-DCFPyL scans. Imaging findings were compared to histopathology as the truth standard.

### Results

For detection of pelvic lymph node metastases in Cohort A (n=252 evaluable), ¹⁸F-DCFPyL PET/CT imaging had a median sensitivity of 40.3% (95% CI: 28.1-52.5%), median specificity of 97.9% (95% CI: 94.5-99.4%); and median PPV and NPV of 86.7% (95% CI: 69.7-95.0%) and 83.2% (95% CI: 78.0-88.0%), respectively. In Cohort B (n=93 evaluable), median sensitivity and PPV were 95.8% (95% CI: 87.8-99.0%) and 81.9% (95% CI: 73.7-90.2%), respectively. Sensitivity and PPV of ¹⁸F-DCFPyL PET/CT were evaluated in different lesion locations on a region level (prostatic, pelvic and extra-pelvic) in Cohort B. Median sensitivity and PPV for the pelvic region were 100% (95% CI: N/A) and 79.5% (95% CI: 67.0-92.0%), respectively; and median sensitivity and PPV for the extra-pelvic region were 94.9% (95% CI: 82.0-99.0%) and 86.1% (95% CI: 76.0-96.0%), respectively. There were no evaluable subjects with a local recurrence to the prostatic region for analysis. Individual reader assessments are shown in the table below. No drug-related serious adverse events were observed. Twenty-seven (7.0%) men experienced ≥1 drug-related adverse event, with dysgeusia (2.1%) and headache (2.1%) being most frequent.

### Conclusions

¹⁸F-DCFPyL PET/CT has a favorable toxicity profile and is generally well tolerated in patients with PCa. ¹⁸F-DCFPyL PET/CT demonstrated high sensitivity in reliably detecting distant metastatic prostate cancer and high specificity in confirming the absence of pelvic lymph node metastases. The associated strong PPV and NPV of ¹⁸F-DCFPyL imaging in these disease settings indicate its potentially high clinical utility. ¹⁸F-DCFPyL is currently under investigation in a phase 3 study in patients with biochemical recurrence of PCa.

TABLE 3
Diagnostic Performance by Region (Cohort B)

| Region | Reviewer | Sensitivity | | PPV |
|---|---|---|---|---|
| Prostate bed | — | — | | — |
| Pelvic | 1 | 100% | | 76.2% (0.63, 0.89) |
| | 2 | 96.9% | (0.82, 1.0) | 79.5% (0.67, 0.92) |
| | 3 | 100% | | 88.9% (0.78, 0.99) |
| Extra-pelvic | 1 | 97.4% | (0.85, 1.0) | 86.1% (0.76, 0.96) |
| | 2 | 94.9% | (0.88, 0.99) | 84.1% (0.73, 0.95) |
| | 3 | 86.8% | (0.72, 0.95) | 86.8% (0.76, 0.97) |

### Example 4

### Diagnostic Performance of ¹⁸F-DCFPyL in the OSPREY Trial: A Prospective Phase 2/3 Multi-Center Study of ¹⁸F-DCFPyL PET/CT Imaging in Patients (Pts) with Known or Suspected Metastatic Prostate Cancer (mPC)

### Background

Accurate detection of prostate cancer is imperative to patient management, yet standard imaging methods perform poorly in accurately detecting mPC. ¹⁸F-DCFPyL is a novel PET imaging agent that selectively binds to prostate-specific membrane antigen, a recognized target for prostate cancer. OSPREY was a prospective, multicenter study in pts with either newly diagnosed high-risk prostate cancer (cohort A), or known or suspected mPC (cohort B). Here we focus on Cohort B.

### Methods

117 men planned for biopsy of recurrent or mPC received ¹⁸F-DCFPyL. Pts underwent image-guided biopsy. Sensitivity, positive predictive value (PPV), and safety of ¹⁸F-DCFPyL PET/CT were the key endpoints for Cohort B. ¹⁸F-DCFPyL PET/CT scans were evaluated by three independent, blinded central readers; and results were compared to histopathology as the truth standard.

### Results

The sensitivity and PPV of ¹⁸F-DCFPyL PET/CT as compared to histopathology ranged from 92.9-98.6% (lower bound of 95% CI: 84.0-91.6%) and 81.2-87.8%, respectively. Diagnostic performance by anatomic location showed high sensitivity and high PPV in all sites of disease (see table below). Only two (1.7%) cohort B pts experienced ≥1 drug-related AE (dysgeusia and generalized rash), both were mild (Grade 1) in severity.

### Conclusions

¹⁸F-DCFPyL PET/CT was well tolerated and demonstrated high sensitivity and PPV in accurately detecting nodal, bone, and visceral/soft tissue metastases. A positive ¹⁸F-DCFPyL PET/CT scan is highly likely to represent pathologically proven distant disease, demonstrating the potential of ¹⁸F-DCFPyL as a PET imaging agent to favorably influence treatment planning.

TABLE 4

| | | Sensitivity | | PPV | |
|---|---|---|---|---|---|
| | | Range for 3 | | Range for 3 | |
| | N | readers (%) | | readers (%) | |
| Anatomic | Evalu- | (lower bound of | | (lower bound of | |
| Location | able | 95% CI (%)) | | 95% CI (%)) | |
| All tissues | 92-93 | 92.9-98.6 | (84.0-91.6) | 81.2-87.8 | (72.9-80.0) |
| Bone | 43-44 | 90.3-96.9 | (74.0-82.9) | 78.9-82.3 | (66.0-69.5) |
| Lymph | 39 | 93.3-100 | (77.6-86.0) | 80.0-90.6 | (66.8-75.0) |
| Node | | | | | |
| Viscera/ | 10 | 88.9-100 | (54.0-65.5) | 90.0-100 | (57.0-62.0) |
| Soft | | | | | |
| Tissue | | | | | |

1. A method of treating, or making a treatment management decision for, prostate cancer in a subject, the method comprising:
determining the presence or absence of one or more metastases in the subject with 18F-DCFPyL PET/CT, and
treating or making a treatment management decision for the subject based on the presence or absence of one or more metastases,
wherein the subject is one with metastatic or recurrent prostate cancer, or is suspected of having metastatic or recurrent prostate cancer.

2. The method of claim 1, wherein the subject is one with extra-prostatic lesions, or is suspected of having extra-prostatic lesions.

3. The method of claim 1, wherein the subject is one with pelvic metastases, or is suspected of having pelvic metastases.

4. The method of claim 1, wherein the subject is one with distant metastases, or is suspected of having distant metastases.

5. The method of claim 1, wherein the subject is one with nodal, bone, and/or visceral/soft tissue metastases.

6. The method of claim 1, wherein one or more of the metastases is greater than 4 mm.

7. The method of claim 6, wherein the mean or median lymph node metastases is ≥4 mm.

8. The method of claim 1, wherein 18F-DCFPyL is administered 1-2 hours prior to PET/CT to the subject.

9. The method of claim 1, wherein 9 mCi (333 MBq) of 18F-DCFPyL is administered by IV injection to the subject.

10. The method of claim 1, wherein the method further comprises treating the subject based on the determining.

11. The method of claim 10, wherein the treatment is any one of the treatments provided herein.

12. The method of claim 1, wherein the making a treatment management decision includes any one or more of the treatment management decisions provided herein.

13. The method of claim 1, wherein the subject is any one of the subjects as described herein.

14. The method of claim 1, wherein the subject has undergone a prior diagnostic test, such as a PSA test or a conventional imaging test.

15. The method of claim 14, wherein the prior diagnostic test was negative or equivocal findings for prostate cancer on conventional imaging.

16. The method of claim 1, wherein the method further comprises a step of performing an additional diagnostic test on the subject, such as a PSA test or conventional imaging test.

17. The method of claim 16, wherein the PSA test result was detectable or rising PSA post-radical prostatectomy in the subject.

18. The method of claim 16, wherein the PSA test result was an increase in PSA level post-radiation therapy, cryotherapy, or brachytherapy in the subject.
